(19) United States
(12) Patent Application Publication
(10) Pub. No.: US 2011/0015519 A1
(43) Pub. Date: Jan. 20, 2011

(54) MEDICAL SYSTEM AND METHOD FOR THE POSITIONALLY CORRECT ASSOCIATION OF AN IMAGE DATA SET WITH AN ELECTROMAGNETIC NAVIGATION SYSTEM

(76) Inventor: Rainer Graumann, Hochstadt (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/920,781

(22) PCT Filed: Mar. 2, 2009
(86) PCT No.: PCT/EP2009/052464

§ 371 Date: Sep. 2, 2010

(30) Foreign Application Priority Data
Mar. 6, 2008 (DE) 10 2008 012 857.0

(51) Int. Cl. A61B 6/00

(52) U.S. Cl. 600/424

(57) ABSTRACT

In a method and medical system for positionally correct association of an image data set of a patient, obtained with the medical system, and an N-coordinate system of an electromagnetic navigation system, before a medical procedure at least one sensor coil is attached to the imaging system, and a transformation matrix between the image data set and the sensor coil is determined. During the medical procedure, the image data set is acquired, and the current position of the sensor coil is determined. The image data set is associated in a positionally correct manner with the N-coordinate system based on the known position of the sensor coil relative to the imaging system, and the transformation matrix.

### BACKGROUND OF THE INVENTION

1. Field of the Invention

The invention concerns a medical system and a method for positionally accurate association of an image data set with an electromagnetic navigation system.

2. Description of the Prior Art

Within the scope of a medical procedure—for example an endoscopy, biopsy or surgery—it is known to implement the procedure with the aid of a surgical navigation based on an electromagnetic navigation system and intraoperative imaging. An imaging system used for this is purpose, for example, a mobile x-ray C-arm for 2D or 3D imaging. The image data set is produced in a B-coordinate system of the imaging system. However, the navigation of surgical instruments occurs in an N-coordinate system of the navigation system which thus represents a different reference system. For a positionally accurate association of the image data set created with the imaging system during the operation with the N-coordinate system of the electromagnetic navigation system, presently complicated and error-prone registration procedures must be used. For example, these must be implemented by means of anatomical landmarks of the patient, or at the patient to be treated. Such time-consuming registration procedures lead to a reduction of the acceptance of navigated measures or, respectively, navigation-assisted medical systems. Two registration procedures are presently known, namely with anatomical or radiological landmarks:

For registration by means of anatomical landmarks, at least three landmarks are identified in the preoperative image data sets acquired from the patient and associated with the corresponding anatomical points on the body during the medical procedure. For this purpose the corresponding points or landmarks on the body of the patient are occupied, for example by means of an indicator (thus a navigated pointer) that can be detected by the navigation system.

Given the use of x-ray markers, these must initially be linked in a defined manner with the electromagnetic navigation system, meaning that their spatial position in the N-coordinate system of the navigation system must be known. At least two 2D projection exposures of these x-ray markers, in which the x-ray markers must be visible, are then produced with known acquisition geometry of the imaging system during the medical procedure. The registration is implemented based on the 2D image data and the known position. In an alternative variant, 3D image data of the patient are generated. The x-ray markers must be located in a reconstructed volume of the 3D image data set.

### SUMMARY OF THE INVENTION

An object of the present invention to provide an improved medical system and an improved method for positionally accurate association of an image data set with an electromagnetic navigation system.

This object is achieved by a method in which an image data set that is generated from a patient during a medical procedure is associated with positional accuracy with an N-coordinate system of an electromagnetic navigation system. The imaging system has a B-coordinate system that describes the spatial coordinates of the image data set. According to the invention, before the actual medical procedure—thus before the beginning, for example—at the imaging system at least one sensor coil of the navigation system is placed in a known position relative to the B-coordinate system of said imaging system. The sensor coil then has known spatial coordinates in the B-coordinate system, which also serves for the later image generation via the imaging system. This B-coordinate system is normally selected fixed relative to its matrix (for example in the case of a mobile x-ray C-arm apparatus) which is why the sensor coil is then mounted stationary at the actual C-arm or the base support, for example. As required, the relative spatial position between sensor coil and B-coordinate system is known, even if movable machine parts are involved. For example, this is the case if multiple sensor coils are distributed on a base body and part of the imaging system moving relative to the base body.

Furthermore, before the medical procedure a transformation matrix between the sensor coil and the image data set (thus the B-coordinate system) is determined in a calibration procedure. In other words, a transformation matrix between navigation system and image data set is determined. A positionally accurate association between each image data set that can be generated by the imaging system and the sensor coil (and therefore with the current B-coordinate system) is ensured. According to the invention, a consideration of all relevant metallic bodies in the region of the navigation system ensues. The imaging system (for example) normally has metallic parts that interfere with the spatial accuracy of the navigation system with regard to the sensor coil. For the given configuration of a medical system, such a spatially accurate association possibility thus results between sensor coil (thus the B-coordinate system) and the navigation system and its N-coordinate system) under consideration of the metallic interference bodies.

Multiple sensor coils are normally attached to the imaging system (for example to the C-arm, x-ray head and x-ray detector and to the matrix) in order to be able to redundantly detect a measurement of respectively 5 to 6 degrees of freedom of the imaging system with the aid of the navigation system.

According to the invention, during the medical procedure (i.e. while the patient lies stationary, fixed with regard to the N-coordinate system, for example on a patient bed in a treatment space) the intraoperative image data set of the patient that is to be associated is created with the imaging system, the current position of the sensor coil is determined in the N-coordinate system, and using the predetermined transformation matrix the image data set is associated with positional accuracy with the N-coordinate system.

The method according to the invention thus represents a markerless registration method for positionally accurate association of the image data set in the navigation system. The decisive step is the integration of a sensor coil in the imaging system instead of positioning this at the patient, linked with a suitable calibration and association procedure. The registration procedure that is to be conducted for a medical procedure to be implemented is significantly simplified and therefore designed to be faster and more certain. An integration of navigation-assisted procedure in connection with an electromagnetic navigation system can be integrated more simply into the surgical workflow. The acceptance of the complete method is improved.

In the production of the image data set during the procedure on the patient the position of possible markers does not need to be noted, as in previous known methods. An image data set of the patient that is optimally placed and relevant to the medical procedure can thus be created. The medical procedure is thus accelerated and the dose exposure of the patient is decreased.

The cited calibration procedure must normally ensue only once for a given imaging system or, respectively, medical sis, for example after its manufacture or, respectively, attachment of the sensor coil. However, normally such a calibration is conducted once per year.

In an embodiment of the method, the generation of the image data set and the position determination of the sensor coil in the N-coordinate system ensues simultaneously during the medical procedure. Since the position determination thus ensues during the image acquisition, the entire method is accelerated.

In a further embodiment of the method, the imaging system is removed from the patient during the procedure and after generation of the image data set of the patient. The image data set can be registered with positional accuracy after its acquisition and the detection of the position of the sensor coil in the N-coordinate system of the navigation system. The navigation-assisted implementation of the procedure then no longer requires the imaging system and the sensor coil. The access to the patient is thereby facilitated and improved.

The sensor coil attached to the imaging system requires a return path to the navigation system given operation of the navigation system. In an alternative embodiment of the invention, the sensor coil is a sensor coil that can be read out without wires. The return path is then executed wirelessly, and the sensor coil, for example, is supplied with energy necessary for this by the imaging system. In an alternative embodiment, however, a plug contact with which the sensor coil is connected can also be present at the imaging system. The plug contact can be connected with the navigation system. The sensor coil is thus ultimately connected with the navigation system via the plug contact. The connection between sensor coil and navigation coil can thus be established and released simply via the plug contact. This is in particular of interest if a plurality of sensor coils are non-transiently attached firmly to the imaging system and this should be removed immediately after the imaging.

According to the invention, interfering metal parts are detected and accounted for in the navigation system in order to again establish a high positional accuracy of the navigation system. The consideration of metallic bodies in the region of the navigation system can ensue in different ways: for example, a reference measurement is possible given the presence of the interfering metals in order to detect these by means of measurement technology in an installed navigation system and sensor coils, and it is possible to store this reference measurement in a correspondingly corrected first transformation matrix. A theoretical consideration of the corresponding metal bodies is also conceivable, for example via simulation of the navigation system on a FEM basis.

However, in order to reduce the influence of ferromagnetic parts of the imaging system on the sensor coil or its spatial precision in advance, in an advantageous embodiment of the invention the sensor coil is attached to a pivot arm, wherein the pivot arm is attached to the imaging system and exhibits a predeterminable pivot position. The sensor coil on the (normally non-metallic) pivot arm is then brought into the predeterminable pivot position before the location of the sensor coil. This pivot position is normally chosen so that the sensor coil exhibits a sufficient distance from the metallic parts of the imaging system. In this pivot position the spatial position of the sensor coil is then known again or, respectively, predeterminable relative to the imaging system, and therefore to its B-coordinate system. The effect of this iron mass (which effect interferes with the positional accuracy) is reduced by pivoting the sensor coil away from the imaging system. Here as well a combination with a registration method is possible that also takes into account the iron mass (which is still active, if less so) in the pivoted-away state in the aforementioned calibration procedure.

In the calibration procedure the spatial relationship and the bearing relationship between the image data set generated by the imaging system and the sensor coil is determined. If the at least one aforementioned sensor coil is designated as a first sensor coil at the imaging system, in an advantageous embodiment a second sensor coil of the navigation system is then attached to a calibration body in the calibration procedure. An image data set of the calibration body is thereupon generated and the current position of first and second sensor coil is registered in the N-coordinate system. The position detection of the calibration body in space and in the N-coordinate system then ensues via the second sensor coil; the detection of the B-coordinate system ensues via the first sensor coil. The second transformation matrix can then be generated after producing the calibration image data set.

The above object also is achieved in accordance with the invention by a system having an imaging system that has a B-coordinate system and serves to generate an image data set of a patient during a medical procedure. The medical system furthermore has an electromagnetic navigation system that has an N-coordinate system and at least one sensor coil attached to the imaging system. The sensor coil is attached to the imaging system in a known position relative to the B-coordinate system.

The medical system according to the invention was already explained in connection with the method according to the invention, together with its advantages.

Various embodiments of the medical system together with advantages resulting therefrom have already explained in connection with the method according to the invention.

### BRIEF DESCRIPTION OF THE DRAWINGS

FIG. 1 shows a medical system according to the invention before implementation of a medical procedure.

FIG. 2 shows the medical system of FIG. 1 during the implementation of a medical procedure.

### DESCRIPTION OF THE PREFERRED EMBODIMENTS

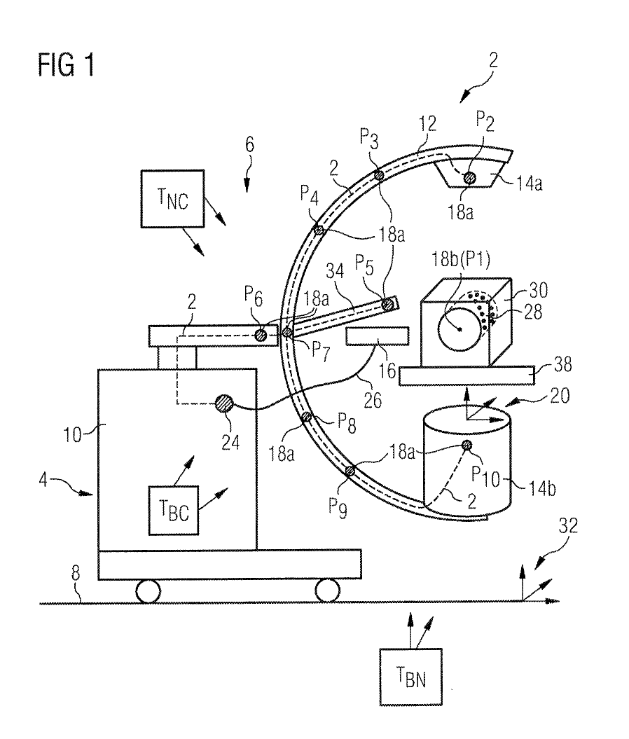

FIG. 1 shows a medical system 2 having an x-ray apparatus 4 as an imaging system and an electromagnetic navigation system 6. The x-ray apparatus 4 has a base support 10 that can be moved in a treatment space 8 and a C-arm 12, attached to the base support 10, with x-ray source 14a and x-ray detector 14b.

The navigation system 6 has a field generator 16—which is held (not shown) fixed in space relative to the treatment space 8—and multiple sensor coils 18a as first sensor coils.

Some of the sensor coils 18a are attached to the C-arm 12 since what is known as a B-coordinate system 20 is fixed stationary to the C-arm 12 as an imaging coordinate system. Therefore the sensor coils 18a just addressed are at rest in the B-coordinate system 20.

All sensor coils 18a are connected via connection lines 22 with a plug contact 24 mounted on the base support 10. The sensor coils 18a can in turn be connected with the field generator 16 via the plug contact 24 and a plug conductor 26. In a variant of the invention that is not shown, a wireless connection is provided here.

In the preoperative situation shown in FIG. 1, a 3D calibration body 28 is introduced into the x-ray apparatus 4, to which 3D calibration body 28 a sensor coil 18b is likewise attached as a second sensor coil. The calibration body is supported on a patient bed 38. In the shown situation, an image data set in the form of a reconstructed 3D volume 30, which contains the 3D calibration body 28 is acquired by the x-ray apparatus 4. It thus also contains the sensor coil 18b. At the same time the spatial position P₁₋₁₀ of the sensor coils 18a,b in the N-coordinate system 32 that is stationary relative to the field generator 16 or treatment space 8 is determined by the field generator 16 or a navigation computer (not shown) contained therein. A transformation matrix T_{BC} between the 3D volume 30 and the C-arm 12 or the sensor coil 18a can be determined in a known manner (not explained in detail here) from the knowledge of the spatial position P₁₋₁₀ and the bearing of the sensor coil 18b in the 3D volume 30. The transformation matrix T_{BC} represents a product T_{BC}=T_{BN}*T_{NC} of the transformation matrix T_{NC} between the C-arm 12 and the field generator 16 and the transformation matrix T_{BN} between the field generator 16 and the 3D volume 30. In other words, the geometric relationships between B-coordinate system 20 and N-coordinate system 32 are expressed by the transformation matrices.

In addition to fixed sensor coils 18a, thus sensor coils 18a directly arranged on the C-arm 12, FIG. 1 also alternatively shows such a sensor coil which is attached on a pivotable arm 34 which is in turn attached to the C-arm 12. Due to the non-metallic execution of the arm 34, this sensor coil 18a with the position P₅ therefore lies at a distance from the metallic body of the x-ray apparatus 4. The position P₅ can therefore be determined exactly by the field generator 16 without additional cost. To determine the positions P₂₋₄ and P₆₋₁₀ whose associated sensor coils 18a respectively rest directly on a metallic part of the C-arm 12, in FIG. 1 an additional calibration procedure is necessary in order to initially determine the exact transformation matrices T_{NC} and T_{BN} which are initially affected by the influence of the metallic parts of the C-arm 12 in the range of the field generator 16. The patient bed 38 also affects the sensor coils 18a.

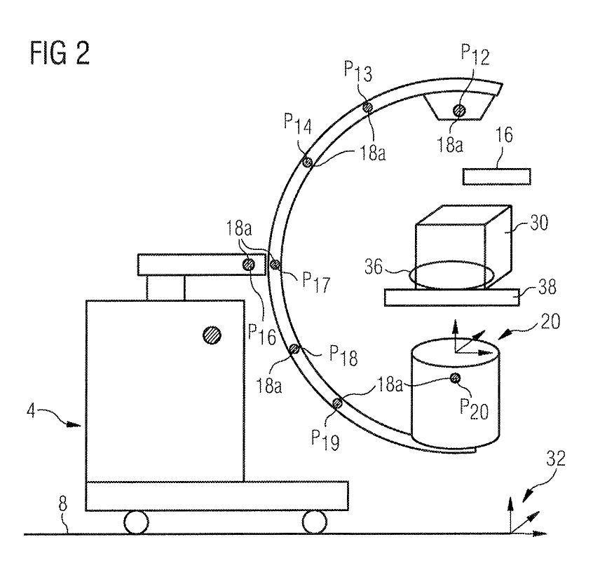

FIG. 2 shows the x-ray apparatus 4 in a medical use, namely in the radioscopy of a patient 36. For this the x-ray apparatus 4 was displaced in the treatment space 8 (thus relative to the N-coordinate system 32), which is why it now assumes a new position relative to this or, respectively, relative to the field generator 16 (which continues to be stationary). In FIG. 2 a 3D volume 30 of the patient 36 is acquired and at the same time the new positions P₁₂₋₂₀ of the sensor coils 18a displaced with the apparatus are determined in the N-coordinate system 32. Given a consistent transformation matrix T_{BC} between 3D volume 30 and x-ray apparatus 4, the 3D volume 30 can also be correctly arranged in the coordinate system 32 using the transformation matrix T_{NC} between the x-ray apparatus 4 and the field generator 16, which is why a medical instrument (not shown) for a procedure on the patient, which medical instrument is likewise oriented in the N-coordinate system 32, can be precisely guided to the desired point in the patient 36 using the 3D volume 30. The cited method can also be implemented for 2D imaging in a variant that is not shown.

In FIG. 2 the arm is expanded from the position shown in FIG. 1—after the intraoperative determination of the position of the C-arm 12—and is no longer visible in order to completely clear the internal space of the C-arm 12 for the patient 36.

Although modifications and changes may be suggested by those skilled in the art, it is the intention of the inventor to embody within the patent warranted heron all changes and modifications as reasonably and properly come within the scope of his contribution to the art.

I claim as my invention:

1.-11. (canceled)

12. A method for spatially accurate association of an image data set of a patient with an N-coordinate system of an electromagnetic navigation system, comprising the steps of:
before acquiring an image data set from a patient using an imaging system having a B-coordinate system and comprising metallic bodies, placing a sensor coil of the navigation system at a known position relative to the B-coordinate system of the imaging system;
also before acquiring said image data set, determining, in a processor, a transformation matrix between the image data set and said sensor coil in a calibration procedure dependent on perturbations caused by said metallic bodies of an electromagnetic field generated by the navigation system, detected by said sensor coil;
placing a patient at rest with respect to said N-coordinate system and acquiring the image data set from the patient with said imaging system during a medical procedure;
also during said medical procedure, determining a current position of the sensor coil in the N-coordinate system; and
in a processor, automatically associating the image data set with the N coordinate system with spatial accuracy using said transformation matrix and said current position of the sensor coil and the known position of the sensor coil relative to the B-coordinate system of the imaging system.

13. A method as claimed in claim 12 comprising acquiring said image data set and determining said current position of the sensor coil simultaneously during said medical procedure.

14. A method as claimed in claim 12 comprising removing said imaging system from the patient during said medical procedure, after acquiring said image data set.

15. A method as claimed in claim 12 comprising connecting said sensor coil with said navigation system via a plug contact at the imaging system.

16. A method as claimed in claim 12 comprising attaching the sensor coil to a pivot arm and attaching the pivot arm to said imaging system, and bringing said pivot arm to a predetermined pivot position to place said sensor coil in said known position relative to said B-coordinate system of the imaging system.

17. A method as claimed in claim 12 wherein said sensor coil at said known position is a first sensor coil, and wherein said navigation system comprises a second sensor coil, and mounting said second sensor coil at a calibration body in said calibration procedure, and obtaining an image data set of said calibration body in said calibration procedure and determining a current position of the first sensor coil and the second sensor coil in the N-coordinate system.

18. A medical system for spatially accurate association of an image data set of a patient with an N-coordinate system of an electromagnetic navigation system, comprising the steps of:
an imaging system having a B-coordinate system and comprising metallic bodies;
a navigation system comprising a sensor coil placed at a known position relative to the B-coordinate system of the imaging system, before acquisition of an image data set from a patient using the imaging system;
a processor configured to determine, also before acquiring said image data set, a transformation matrix between the image data set and said sensor coil in a calibration procedure dependent on perturbations caused by said metallic bodies of an electromagnetic field generated by the navigation system, detected by said sensor coil;
said imaging system being operated to acquire an image data set from a patient at rest with respect to said N-coordinate system, during a medical procedure;
said processor being supplied with a current position of the sensor coil in the N-coordinate system during said medical procedure, and said processor being configured to automatically associate the image data set with the N coordinate system with spatial accuracy using said transformation matrix and said current position of the sensor coil and the known position of the sensor coil relative to the B-coordinate system of the imaging system.

19. A medical system as claimed in claim 18 wherein said image data set is acquired and said current position of the sensor coil is determined simultaneously during said medical procedure.

20. A medical system as claimed in claim 18 wherein said imaging system is removed from the patient during said medical procedure, after acquiring said image data set.

21. A medical system as claimed in claim 18 comprising via a plug contact at the imaging system that connects said sensor coil with said navigation system.

22. A medical system as claimed in claim 18 wherein the sensor coil is attached to a pivot arm and the pivot arm is attached to said imaging system, and wherein said pivot arm is operated to bring said pivot arm to a predetermined pivot position that places said sensor coil in said known position relative to said B-coordinate system of the imaging system.

23. A medical system as claimed in claim 18 wherein said sensor coil at said known position is a first sensor coil, and wherein said navigation system comprises a second sensor coil, and a calibration body to which said second sensor coil is mounted in said calibration procedure, and wherein said imaging system is operated to obtain an image data set of said calibration body in said calibration procedure and said processor is supplied with a current position of the first sensor coil and the second sensor coil in the N-coordinate system.
